(19) United States
(12) Patent Application Publication
(10) Pub. No.: US 2022/0252511 A1
(43) Pub. Date: Aug. 11, 2022

(54) COMPOSITION ANALYZER AND COMPOSITION ANALYSIS SYSTEM

(71) Applicant: MEGA CRYSTAL BIOTECHNOLOGY SINGAPORE PTE. LTD, Singapore (SG)

(72) Inventors: YI-SHENG TING, New Taipei City (TW); YU-TSUNG CHEN, New Taipei City (TW)

(21) Appl. No.: 17/665,171

(22) Filed: Feb. 4, 2022

(30) Foreign Application Priority Data
Feb. 9, 2021 (TW) 110105005

(51) Int. Cl. G01N 21/3563; G01N 33/00

(52) U.S. Cl. CPC G01N 21/3563; G01N 2201/022; G01N 2201/062; G01N 33/0098

(57) ABSTRACT

A composition analyzer has an accommodating device, an optical detection device, a driving device and a support part. The accommodating device accommodates an object to be analyzed, and has an accommodating space, a transparent plate and a rotating part, wherein the transparent plate is disposed on one of two opposite sides of the accommodating space, the rotating part is disposed on the accommodating device. The optical detection device has a solid state light source emitter and a light receiver, wherein another one of the two opposite side of the accommodating space is disposed with one of another one transparent plate, a light reflection plate and a non-transparent plate. The driving device is connected to the rotating part. The support part is pivotally connected to the rotating part. The accommodating device rotates in the YZ plane, which can perform uniform and repeated measurements to analyze content of grains.

### TECHNICAL FIELD

The present disclosure relates to a technical field of a composition analyzer, and in particular to, a composition analyzer and composition analysis system, both of which can perform uniform and repeated measurements.

### RELATED ART

Selection or breeding of grain varieties has always been the direction of agricultural efforts. For the judgment of grains of good varieties, it is nothing more than to judge the value of the grains based on the proportion of components contained in the grains. Nowadays, many advanced countries combine agriculture with modem technology, and thus, intelligent agriculture is established.

In intelligent agriculture, spectrometer is usually used to detect nutrient compositions of the grains, and especially for the buyer with a large amount of grain demand and composition requirement, they pay more attention to the accuracy and the rapidity of data after detection. When analyzing grains by using the current composition analyzer, the grains are placed in the analysis container, and the composition ratio of the grains is analyzed by the spectrometer through the horizontal rotation of the analysis container or a fixed and static manner of the analysis container. The above-mentioned method can only measure the grains at the partial positions in the analysis container, so it is difficult to repeat the measurement for many times to obtain an average value. In addition, the grain composition analyzer described in Japanese Patent Publication No. JP6088770B2 uses a spectroscopic method to quantitatively analyze specific components contained in grains by grain. The analyzer provided by the Japanese patent can measure grains by grain and obtain more realistic values. In practice, however, this is time-consuming and impractical when the grain content to be analyzed is large. In addition, there is a waterfall method in the market to flow the grains from top to bottom, and the measurement is carried out through the spectral method while flowing. This method can improve some of the shortcomings of the composition analyzer mentioned above, but it is not suitable for repeating many times. The repeated measurement of grains can only be carried out manually, so it is still time-consuming and labor-intensive and may contaminate the grains to be tested.

Therefore, the present disclosure is to illustrate how to use means of innovative hardware design to improve the problems of the conventional composition analyzer, such as to achieve balance among the grain content, uniform measurement and repeated measurement. The above problems are still issues that related researchers need to continue to work hard to overcome and solve.

### SUMMARY

To achieve one objective of the present disclosure, the present disclosure is to provide a technical solution to improve the conventional composition analyzer, and the problems of balance among the grain content, uniform measurement and repeated measurement existed in the conventional composition analyzer can be solved.

The present disclosure provides a composition analyzer, comprising an accommodating device, an optical detection device, a driving device and at least a support part. The accommodating device is configured to accommodate an object to be analyzed, and has an accommodating space, a transparent plate and a rotating part, wherein the transparent plate is disposed on one of two opposite sides of the accommodating space, the rotating part is disposed on the accommodating device, an extending direction of the rotating part is defined as an X direction, the X direction is different from a Y direction and a Z direction, the Y direction and the Z direction form a YZ plane, the accommodating device is configured to rotate in the YZ plane, a normal line of the YZ plane and the X direction have an angle therebetween, and the angle is larger than or equal to 0 degrees and less than 90 degrees. The optical detection device has a solid state light source emitter and a light receiver, wherein the solid state light source emitter has a light source, the light receiver receives a light beam emitted from the light source, and the transparent plate is provided to the light beam for passing through, wherein another one of the two opposite side of the accommodating space is disposed with one of another one transparent plate, a light reflection plate and a non-transparent plate. The driving device is connected to the rotating part. The support part is pivotally connected to the rotating part.

In one embodiment, when the other one of the two opposite side of the accommodating space is disposed with other one transparent plate, the solid state light source emitter and the light receiver are respectively disposed on positions adjacent to the two opposite sides of the accommodating device.

In one embodiment, when the other one of the two opposite side of the accommodating space is disposed with the light reflection plate, the solid state light source emitter and the light receiver are disposed positions adjacent to the one of the two opposite sides of the accommodating device.

In one embodiment, when the other one of the two opposite side of the accommodating space is disposed with the a non-transparent plate, the solid state light source emitter is disposed on a position adjacent to the one of the two opposite sides of the accommodating device, and the light receiver is disposed on a position adjacent to the other one of the two opposite sides of the accommodating device and facing to the one of the two opposite sides of the accommodating device.

In one embodiment, the accommodating device further comprises a cover and an opening, the opening is communicated to the accommodating space, and the cover is configured to movably close the opening.

In one embodiment, the cover is sieved to the opening.

In one embodiment, the cover is pivotally connected to the accommodating device via a first pivot.

In one embodiment, material of the transparent plate comprises glass, sapphire, quartz or acrylic.

In one embodiment, a wavelength the light source of the solid state light source emitter is ranged from 180 nm to 2500 nm.

In one embodiment, a wavelength the light source of the solid state light source emitter is ranged from 400 nm to 1700 nm.

In one embodiment, a cross-sectional shape of the accommodating device is a circle, an ellipse, a polygon or an irregular shape.

In one embodiment, the composition analyzer is disposed in an interior of a case.

In one embodiment, the case further comprises a cover, and the cover is pivotally connected to the case via a second pivot.

In one embodiment, the case further comprises at least a heat dissipation hole disposed thereon.

In one embodiment, the case further comprises a heat dissipation unit disposed in the interior of the case.

In one embodiment, the composition analyzer further comprises a sensor.

In one embodiment, the sensor comprises at least one of a relative humidity sensor and a temperature sensor.

The present disclosure provides a composition analysis system adapted to a composition analyzer, and the composition analysis system comprises an accommodating device, an optical detection device, a driving device, at least a support part and a first processor. The accommodating device is configured to accommodate an object to be analyzed, and has an accommodating space, a transparent plate and a rotating part, wherein the transparent plate is disposed on one of two opposite sides of the accommodating space, the rotating part is disposed on the accommodating device, an extending direction of the rotating part is defined as an X direction, the X direction is different from a Y direction and a Z direction, the Y direction and the Z direction form a YZ plane, the accommodating device is configured to rotate in the YZ plane, a normal line of the YZ plane and the X direction have an angle therebetween, and the angle is larger than or equal to 0 degrees and less than 90 degrees. The optical detection device has a solid state light source emitter and a light receiver, wherein the solid state light source emitter has a light source, the light receiver receives a light beam emitted from the light source, and the transparent plate is provided to the light beam for passing through, wherein another one of the two opposite side of the accommodating space is disposed with one of another one transparent plate, a light reflection plate and a non-transparent plate. The driving device is connected to the rotating part. The support part is pivotally connected to the rotating part. The first processor is electrically connected to the optical detection device, the driving device, a grain analysis module, a first wireless communication module and a global positioning system.

In one embodiment, the composition analysis system further comprises a sensor electrically connected to the first processor.

In one embodiment, the sensor comprises at least one of a relative humidity sensor and a temperature sensor.

In one embodiment, the composition analysis system further comprises a first setting unit electrically connected to the first processor, wherein the first setting unit is used to input at least one parameter related to a planted crop.

In one embodiment, the at least one parameter related to the planted crop input by the first setting unit comprises at least one of crop information, a crop kind, a record date, an analysis area and a crop harvesting plan.

In one embodiment, the composition analysis system further comprises a first display device electrically connected to the first processor.

In one embodiment, the first wireless communication module is communicatively connected to a second wireless communication module of an electronic device, and the second wireless communication module is electrically connected to a second processor.

In one embodiment, the electronic device further comprises a second setting unit electrically connected to the second processor, wherein the second setting unit is used to input at least one parameter related to a planted crop.

In one embodiment, the at least one parameter related to the planted crop input by the second setting unit comprises at least one of crop information, a crop kind, a record date, an analysis area and a crop harvesting plan.

In one embodiment, the electronic device further comprises a second display device electrically connected to the second processor.

To sum up, the composition analyzer of the present disclosure utilizes the rotation of the accommodating device in the YZ plane, so that the grains to be analyzed can be detected under the condition of uniform mixing, and the amount of multiple repeated measurements is achieved. At the same time, the value of the detected object to be analyzed can be transmitted to the user's electronic device through the composition analysis system, which is the basis for the user to formulate a crop harvesting plan in the future.

### DESCRIPTIONS OF DRAWINGS

FIG. 1A is a schematic diagram of a composition analyzer according to an embodiment of the present disclosure.

FIG. 1B is a first top view of a composition analyzer according to an embodiment of the present disclosure.

FIG. 1C is a second top view of a composition analyzer according to an embodiment of the present disclosure.

FIG. 1D is a side view of an accommodating device for an object to be analyzed according to an embodiment of the present disclosure.

FIG. 1E is a sectional view of an accommodating device for an object to be analyzed according to an embodiment of the present disclosure.

FIG. 1F is a first operation diagram of an accommodating device for an object to be analyzed according to an embodiment of the present disclosure.

FIG. 1G is a second operation diagram of an accommodating device for an object to be analyzed according to an embodiment of the present disclosure.

FIG. 2 is a radiation spectrum of a light emission diode (LED) according to a first embodiment of the present disclosure.

FIG. 3 is a radiation spectrum of a light emission diode (LED) according to a second embodiment of the present disclosure.

FIG. 4 is a radiation spectrum of a light emission diode (LED) according to a third embodiment of the present disclosure.

FIG. SA is a plot of a time domain signal of an object to be analyzed, which is measured by the optical detection device of present disclosure.

FIG. 5B is a plot of a frequency domain signal of an object to be analyzed after the time domain signal of the object to be analyzed is performed with a Fourier transform.

FIG. SC is a plot of a time domain signal of an object to be analyzed after the frequency domain signal of the object to be analyzed is filtered and performed with an inverse Fourier transform.

FIG. 6 is a spectrum of wheat detected by a composition analyzer of the present disclosure.

FIG. 7 is a spectrum of wheat detected by a composition analyzer of the prior art.

FIG. 8 is a composition comparison analysis table chart of the spectrums of wheat detected by the composition analyzers of the present disclosure and the prior art.

FIG. 9 is a comparison analysis table chart of the spectrums of the object to be analyzed which is detected by the composition analyzers of the present disclosure and the prior art.

FIG. 10 is a block diagram of a composition analysis system according to an embodiment of the present disclosure.

FIG. 11 is a block diagram of an electronic device according to an embodiment of the present disclosure.

FIG. 12 a schematic diagram of a crop origin according to an embodiment of the present disclosure.

FIG. 13 is a first schematic diagram of an analysis area according to an embodiment of the present disclosure.

FIG. 14 is a second schematic diagram of an analysis area according to an embodiment of the present disclosure.

FIG. 15A and FIG. 15B are a schematic diagram of the parameter related to a planted crop, which is input by the first setting unit and the second setting unit according to an embodiment of the present disclosure.

### DETAILS OF EXEMPLARY EMBODIMENTS

In order to facilitate understanding of the technical characteristics, content and advantages of the present disclosure and the effects that can be achieved, the present disclosure is described in detail as follows in conjunction with the accompanying drawings, and the drawings used therein are only for illustration It is not necessarily the real proportion and precise configuration after the implementation of the present disclosure, so it should not be interpreted or limited to the scope of rights of the present disclosure in actual implementation based on the proportion and configuration relationship of the attached drawings.

In order to make the narration of the disclosure of the present disclosure more detailed and complete, an illustrative description is provided below for the embodiments of the present disclosure and specific embodiments; but this is not the only form of implementing or using the specific embodiments of the present disclosure.

In the whole of this specification, as long as the expression in the singular form is not specifically mentioned, it should be understood as also including the concept of the plural form.

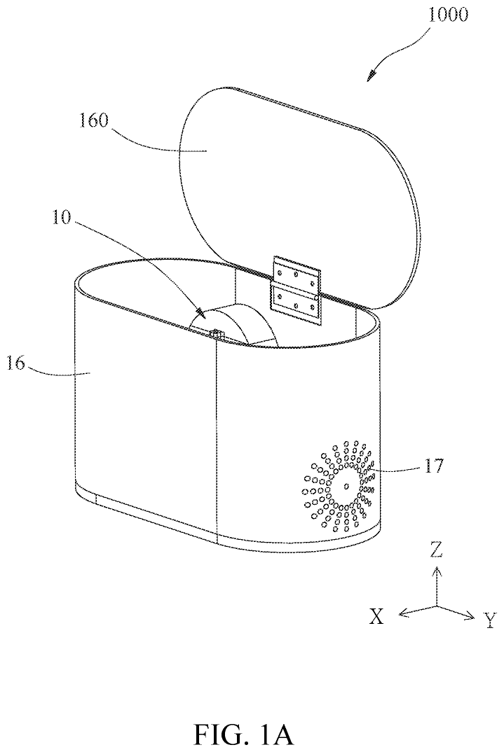

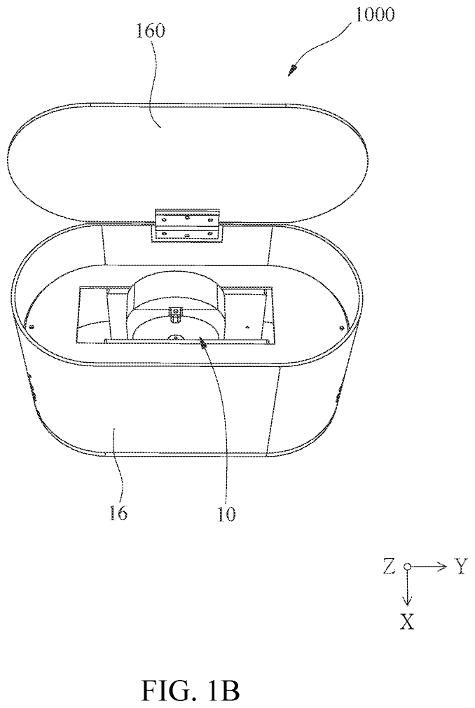

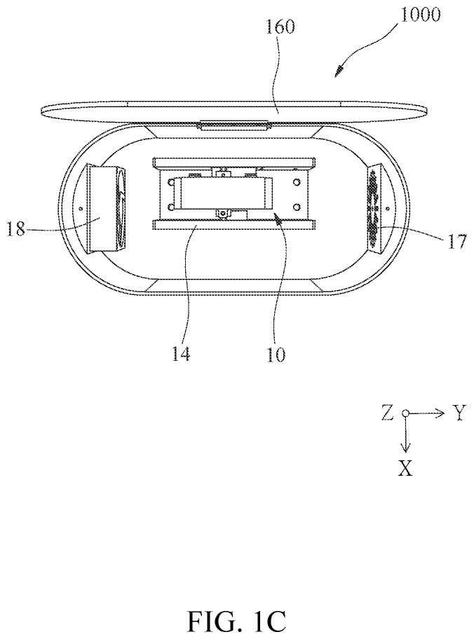

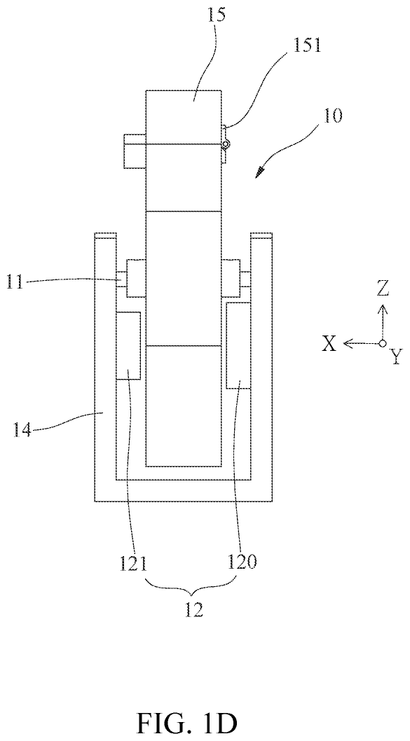

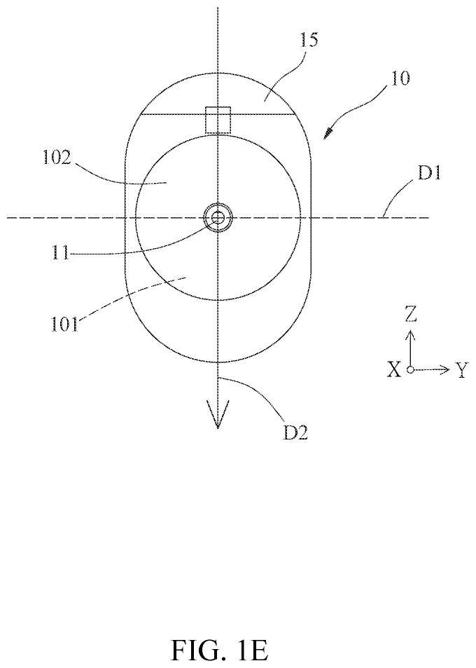

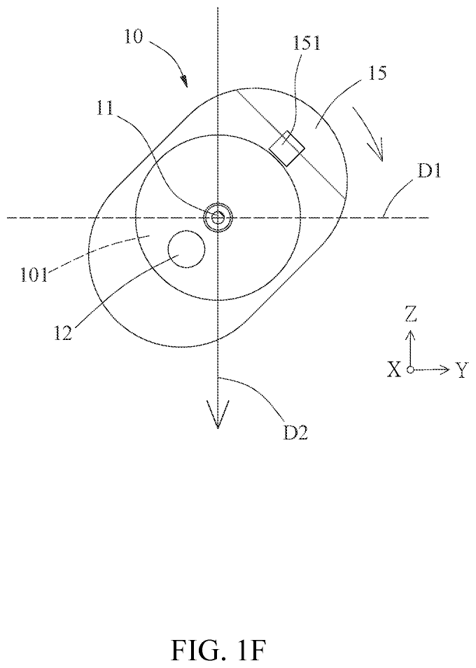

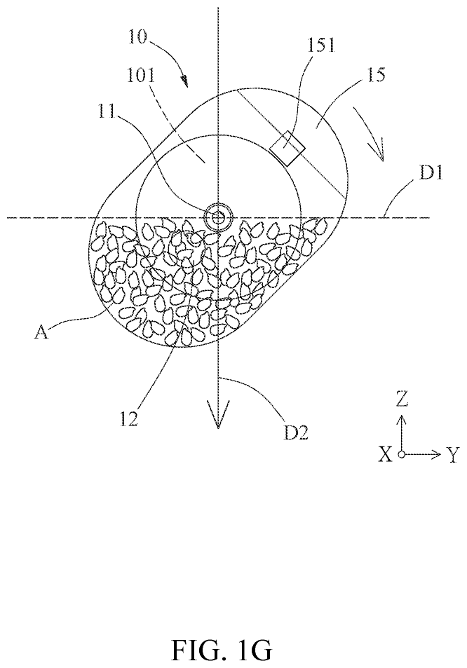

Refer to FIG. 1A through FIG. 1D, FIG. 1A is a schematic diagram of a composition analyzer according to an embodiment of the present disclosure, FIG. 1B is a first top view of a composition analyzer according to an embodiment of the present disclosure, FIG. 1C is a second top view of a composition analyzer according to an embodiment of the present disclosure, and FIG. 1D is a side view of an accommodating device for an object to be analyzed according to an embodiment of the present disclosure. The composition analyzer (1000) comprises an accommodating device (10), an optical detection device (12), a driving device (13) and at least a support part (14), and the accommodating device (10) has an accommodating space (101), at least a transparent plate (102) and a rotating part (11).

The rotating part (11) can be arranged through the accommodating device (10) or be respectively arranged on both sides of the accommodating device (10) to drive the accommodating device (10) to rotate as an axis, or a plurality of the rotating parts (11) are arranged around the accommodating device (10). For example, the rotating part (11) can be a gear which can rotate by meshing with the gear of the accommodating device (10); or, the plurality of the rotating parts (11) can also rotate or not rotate respectively, and the plurality of the rotating parts (11) cooperate with each other to drive the accommodating device (10) to rotate; or, the plurality of rotating parts (11) may also include chains, crawler belts, belts or other objects that can drive the accommodating device (10), so that the object (A) to be analyzed, which is accommodated in the accommodating device (10) can be flipped up and down to achieve a uniform mixing effect in a short time.

The support part (14) is pivotally connected to the rotating part (11). For example, the support part (14) can be pivotally connected to two ends or one end of the rotating part (11) according to actual needs, and the driving device (13) is connected to the rotating part (11). In actual implementation, the driving device (13) drives the rotating part (11) to rotate, the rotating part (11) drives the accommodating device (10) to rotate at the same time, and the support part (14) not only provides the rotating part (11) pivot connection, but also at the same time, the accommodating device (10) is supported so that the accommodating device (10) can be stably rotated, and the rotation speed, frequency and direction of the driving device (13) can adjusted according to the size, quantity and weight of an object (A) to be analyzed (as shown in FIG. 1G). The driving device (13) can be the servo motor for example, and the present disclosure is not limited thereto.

The optical detection device (12) can detect an object (A) to be analyzed, and generate corresponding spectrums of absorption spectrum, transmission spectrum or reflection spectrum, and through the analysis of the spectrums, the relative component ratio of the object (A) to be analyzed can be known. For example, the object (A) to be analyzed in the present disclosure is a grain, and the values of water, protein and gray matter of the grain can be obtained by analyzing the spectrum.

Further, the transparent plate (102) is disposed on one side of the two opposite sides of the accommodating device (10), and material the transparent plate (102) includes glass, sapphire, quartz or acrylic, but the present disclosure is not limited thereto. The transparent plate (102) can allow light sources or specific wavelength light sources to pass through. Another one of the two opposite side of the accommodating space (101) is disposed with one of another one transparent plate (102), a light reflection plate and a non-transparent plate. When the other one of the two opposite side of the accommodating space (101) is disposed with other one transparent plate (102), the light source can pass through the accommodating space (101) from one side of the accommodating device (10) and to the other side of the accommodating device (10).

Refer to FIG. 1D again, and the optical detection device (12) comprises a solid state light source emitter (120) and a light receiver (121). For example, the solid state light source emitter (120) can be a light emitting diode (LED) and a laser diode (LD). The solid state light source emitter (120) has a light source, the light receiver (121) receives a light emitted from the light source, and the transparent plate (102) allows the light to pass through. In one embodiment of the present disclsoure, when the other one of the two opposite side of the accommodating space (101) is disposed with other one transparent plate (102), the solid state light source emitter (120) and the light receiver (121) are respectively disposed on positions adjacent to the two opposite sides of the accommodating device (10). The solid state light source emitter (120) includes a light source. The light source can be, for example, but not limited to, a single light source group or a plurality of sub-light source groups. When the light source includes a plurality of sub-light source groups, each of the sub-light source groups includes a plurality of light-emitting elements, and each emitting light has at least one light-emitting peak wavelength and at least one wavelength range. A plurality of the sub-light source groups and/or a plurality of the light-emitting elements are electrically connected to a circuit board of the light source, and a plurality of the sub-light light source groups are arranged in an irregular shape or in a regular shape. In another one embodiment, when the other one of the two opposite side of the accommodating space (101) is disposed with the light reflection plate, the solid state light source emitter (120) and the light receiver (121) are disposed positions adjacent to the one of the two opposite sides of the accommodating device (10). The solid state light source emitter (120) has a light source, and the light receiver (121) receives a light reflected by the light reflection plate. A light path of the light is formed by the light source, the light reflection plate and the light receiver (121). The light reflection plate can be a whiteboard, a metal plate, a reflective plate, a reflective mirror, a reflective coating or any object with reflective ability. In another one embodiment, when the other one of the two opposite side of the accommodating space (101) is disposed with the a non-transparent plate, the solid state light source emitter (120) is disposed on a position adjacent to the one of the two opposite sides of the accommodating device (10), and the light receiver (121) is disposed on a position adjacent to the other one of the two opposite sides of the accommodating device (10) and facing to the one of the two opposite sides of the accommodating device (10).

In the embodiment, the light receiver (121) receives a light emitted from the light source, and the travel path of the light between the light source and the light receiver (121) forms a light path, the light receiver (121) may be, for example, a photo detector, photodiode, organic photodiode, photomultiplier, photoconducting detector, silicon bolometer, one-dimensional or multi-dimensional photodiode array, one-dimensional or multi-dimensional charge coupled device (CCD) array, one-dimensional or multi-dimensional complementary metal-oxide-semiconductor (CMOS) array, image sensor (19), camera, spectrometer or hyperspectral camera. The object (A) to be analyzed is placed on the path of the light path, and the light path passes through the object (A) or the optical path forms diffuse reflection light on the surface of the object (A); or, the light path forms penetration and reflection of one or more times on the surface and inside of the object (A) to finally generate the diffuse reflection light. The light receiver (121) converts the diffuse reflection light into an image signal, an spectral signal of the object (A), a voltage signal and/or a current signal, and sends the image signal, spectral signal of the object (A), voltage signal and/or current signal to a first processor (20), and the first processor (20) converts the image signal, spectral signal of the object (A), voltage signal and/or current signal to form an image and/or an spectrum of the object (A). In other word, the light receiver (121) includes an image capture device and/or a photodetector electrically connected to the first processor (20), for example, the image capture device can be a camera, CCD or CMOS to convert the light into the image signal, and the light detector may be a spectrometer to convert the light into the spectral signal of the object (A). In another example, the aforementioned photodiode can convert the light into the voltage signal or the current signal.

Refer to FIG. 1E, and FIG. 1E is a sectional view of an accommodating device for an object to be analyzed according to an embodiment of the present disclosure. An extending direction of the rotating part (11) is defined as an X direction, the X direction is different from a Y direction and a Z direction, and the Y direction and the Z direction form an YZ plane. In practice, a normal line of the YZ plane and the X direction have an angle therebetween, and the angle is larger than or equal to 0 degrees and less than 90 degrees. The accommodating device (10) is configured to rotate in the YZ plane, to make the object (A) accommodated in the accommodating space (101) be flipped up and down, so as to achieve the effect of mixing uniformly in a short time. In an embodiment of the present disclosure, In an embodiment of the present disclosure, the X direction, the Y direction and the Z direction are perpendicular to each other, and a first direction line (D1) is defined to pass through the center of the rotating part (11) and be horizontal to the Y direction. Furthermore, a second direction line (D2) is defined to pass through the center of the rotating part (11) and to be perpendicular to the Y direction and horizontal to the Z direction.

Refer to FIG. 1F and FIG. 1G, FIG. 1F is a first operation diagram of an accommodating device for an object to be analyzed according to an embodiment of the present disclosure, and FIG. 1G is a second operation diagram of an accommodating device for an object to be analyzed according to an embodiment of the present disclosure. The accommodating device (10) can rotate in the YZ plane, and the angle between the normal line of the YZ plane and the X direction is greater than or equal to 0 degrees and less than 90 degrees. In an embodiment of the present disclosure, the accommodating device (10) can accommodate the object (A) to be analyzed, and the object (A) to be analyzed can occupy a certain proportion of the volume of the accommodating space (101), so that when the accommodating device (10) rotates in the YZ plane, the object (A) accommodated in the accommodating space (101) can be flipped up and down to achieve the effect of uniform mixing in a short time.

Refer to FIG. 1G again, and in the embodiment, the solid state light source emitter (120) and the light receiver (121) of the optical detection device (12) are respectively disposed on positions adjacent to the two opposite sides of the accommodating device (10), on which the two transparent plate (102) are disposed. The optical detection device (12) can adjust the position of the optical detection device (12) adjacent to the transparent plates (102) according to the rotating direction of the accommodating device. In an embodiment of the present disclosure, The first direction line (D1) intersects with the second direction line (D2), and the accommodating device (10) is divided into an upper left area, lower left area, upper right area and lower right area. When the accommodating device (10) is rotated clockwise in the YZ plane, the object (A) to be analyzed is distributed in the lower left area or the lower right area. Therefore, the optical detection device (12) can adjust and arrange the lower left area or the lower right area, so that the optical detection device (12) can obtain a better spectrum when detecting the object (A) to be analyzed, so as to facilitate the subsequent analysis of the spectrum.

In an embodiment of the present disclosure, the accommodating device (10) further includes a cover (15) and an opening, the opening communicates with the accommodating space (101), and the cover (15) is movably sealing the opening. In actual implementation, through the opening, the different objects (A) to be analyzed are placed in the accommodating space (101) by the user, and then the cover (15) can be sieved to the opening, or the cover (15) can be pivotally connected to the accommodating device (10) via a first pivot (151), so that the cover (15) can be pivoted to adjust the angle, and finally the cover (15) seals the opening, preventing object (A) to be analyzed from falling out when the accommodating device (10) rotates in the YZ plane.

In one embodiment of the present disclosure, the cross-sectional shape of the accommodating device (10) is any shape, such as a circle, an ellipse, a polygon or an irregular shape, which can be beneficial to make the object (A) to be analyzed and can be mixed uniformly, and the present disclosure is not limited thereto.

In one embodiment of the present disclosure, the composition analyzer (1000) is further arranged inside a case (16), the case (16) can provide anti-collision, anti-drop or anti-scratch protection to protect the composition analyzer (1000), and the size, shape or color of the case (16) can be adjusted according to the user's needs, for example, demand of easily carrying. At least one heat dissipation hole (17) is set on one side of the case (16) or a heat dissipation unit (18) is set inside the case (16). The heat dissipation unit (18) can be exemplified but not limited to active heat dissipation fan or heat dissipation sheet, thermal sheet, thermal paste or thermal gel used for passive heat dissipation. When the composition analyzer (1000) is running and the heat dissipation unit (18) used is a fan, it can drive the external air into the case (16), the heat generated when the composition analyzer (1000) operates is conducted out through the heat dissipation hole (17) along with the airflow to provide heat dissipation. The case (16) further includes a cover (160), and the cover (160) is pivotally connected to the case (16) by a second pivot (161), so that the cover (160) can be pivoted to adjust the angle.

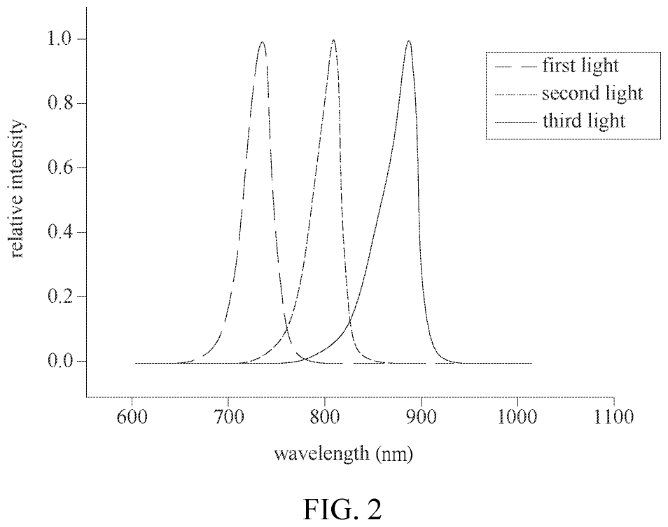

Refer to FIG. 2, the wavelength ranges of the two adjacent light-emitting diodes corresponding to the light-emitting peak wavelengths partially overlap to form a continuous wavelength range wider than the wavelength range of each of the light-emitting diodes, and the continuous wavelength range is between 180 nm and 2500 nm. In FIG. 2, there are three light-emitting peak wavelengths and three corresponding wavelength ranges, which are the first wavelength range corresponding to a first light-emitting peak wavelength (734 nm) of a first light, the second wavelength range corresponding to a second light-emitting peak wavelength (810 nm) of a second light, and the third wavelength range corresponding to a third light-emitting peak wavelength (882 nm) of a third light. The first light-emitting peak wavelength and the second light-emitting peak wavelength are two adjacent light-emitting peak wavelengths, and similarly the second light-emitting peak wavelength and the third light-emitting peak wavelength are also adjacent two light-emitting peak wavelengths. The first wavelength range corresponding to the first light-emitting peak wavelength is between 660 nm and 780 nm, and the second wavelength range corresponding to the second light-emitting peak wavelength of the second light is between 710 nm and 850 nm. The first wavelength range and the second wavelength range partially overlap between 710 nm and 780 nm, so the first wavelength range and the second wavelength range together form the continuous wavelength range between 660 nm and 850 nm. Similarly, the second wavelength range corresponding to the second light-emitting peak wavelength is between 710 nm and 850 nm, and the third wavelength range corresponding to the third light-emitting peak wavelength of the third light is between 780 nm and 940 nm, and the second wavelength range and the third wavelength range partially overlap between 780 nm and 850 nm, so the second wavelength range and the third wavelength range together form the continuous wavelength range between 710 nm and 940 nm. In the present disclosure, the overlapping portions of the wavelength ranges of the two light-emitting diodes corresponding to the adjacent two light-emitting peak wavelengths should preferably be less. Certainly, the wavelength ranges of the two light-emitting diodes corresponding to the adjacent two light-emitting peak wavelengths may not overlap, which will be described later.

The differences between the two adjacent two light-emitting peak wavelength is greater than or equal to 0.5 nm, preferably between 1 nm and 80 nm, more preferably between 5 nm and 80 nm. In FIG. 2, the adjacent first light-emitting peak wavelength (734 nm) and the second light-emitting peak wavelength (810 nm) differ from each other by 76 nm, and the adjacent second light-emitting peak wavelength (810 nm) and the third light-emitting peak wavelength (882 nm) differ from each other by 72 nm. Unless otherwise specified, the limitation of the numerical range described in the present disclosure and the claim scope of the present disclosure always includes the end value. For example, the differences between the two adjacent two light-emitting peak wavelength is between 5 nm and 80 nm, which means greater than or equal to 5 nm and less than or equal to 80 nm.

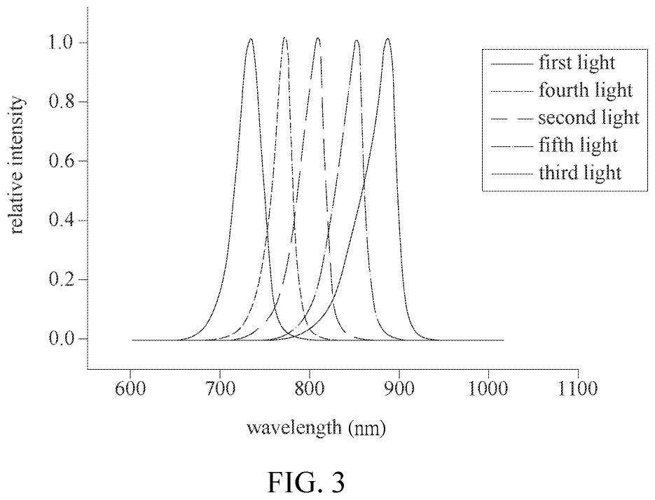

Refer to a second embodiment of FIG. 3, the second embodiment is a derivative embodiment of the first embodiment, and therefore the similarities between the second embodiment and the first embodiment will not be repeated. The difference between the second embodiment and the first embodiment is that the light source of the second embodiment includes five light-emitting diodes, which are respectively the first light-emitting diode, a fourth light emitting diode for radiation with a fourth wavelength range, the second light emitting diode, a fifth light emitting diode for radiation with a fifth wavelength range and the third light emitting diode. There is a fourth light-emitting peak wavelength (772 nm) within the fourth wavelength range, and there is a fifth light-emitting peak wavelength (854 nm) within the fifth wavelength range. In FIG. 3, the wavelengths of the light-emitting peaks are the first light-emitting peak wavelength (734 nm), the fourth light-emitting peak wavelength (772 nm), the second light-emitting peak wavelength (810 nm), the fifth light-emitting peak wavelength (854 nm) and the third light-emitting peak wavelength (882 nm).

The first light-emitting peak wavelength (734 nm) and the fourth light-emitting peak wavelength (772 nm) differ from each other by 38 nm, the fourth light-emitting peak wavelength (772 nm) and the second light-emitting peak wavelength (810 nm) differs from each other by 38 nm, the second light-emitting peak wavelength (810 nm) and the fifth light-emitting peak wavelength (854 nm) differ from each other by 44 nm, and the fifth light-emitting peak wavelength (854 nm) and the third light-emitting peak wavelength (882 nm) differ from each other by 28 nm.

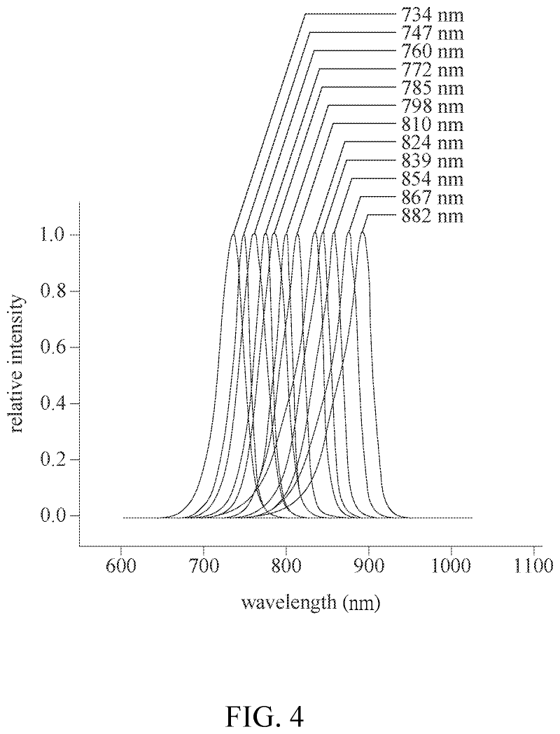

Refer to a third embodiment of FIG. 4, the third embodiment is a derivative embodiment of the first embodiment and the second embodiment, and therefore the similarities between the third embodiment and the second embodiment and the first embodiment will not be repeated. The difference between the third embodiment and the first embodiment is that the light source of the third embodiment includes twelve light-emitting diodes. In FIG. 4, from big to small, the light-emitting peak wavelengths of the twelve light-emitting diodes are 734 nm (the first light-emitting peak wavelength), 747 nm, 760 nm, 772 nm (the fourth light-emitting peak wavelength), 785 nm, 798 nm, 810 nm (the second light-emitting peak wavelength), 824 nm, 839 nm, 854 nm (the fifth light-emitting peak wavelength), 867 nm and 882 nm (the third light-emitting peak wavelength). Among the light-emitting peak wavelengths of the twelve light emitting diodes, each two adjacent light-emitting peak wavelengths differ from each other by 13 nm, 13 nm, 12 nm, 13 nm, 13 nm, 12 nm, 14 nm, 15 nm, 15 nm, 13 nm and 15 nm, respectively. If the light-emitting element in the first embodiment, the second embodiment and the third embodiment is replaced by a laser diode, the wavelength difference between the two adjacent light-emitting peaks can be greater than or equal to 0.5 nm, for example, 1 nm.

The half-height wavelength width corresponding to at least a part of the light-emitting peak wavelengths is greater than 0 nm and less than or equal to 60 nm. Preferably, the half-height wavelength width corresponding to each of the light-emitting peak wavelengths is greater than 0 nm and less than or equal to 60 nm. For example, in the first through third embodiment, from small to big, the light-emitting peak wavelengths are 734 nm (the wavelength of the first light-emitting peak), 747 nm, 760 nm, 772 nm (the wavelength of the fourth light-emitting peak), 785 nm, 798 nm, 810 nm (the wavelength of the second light-emitting peak), 824 nm, 839 nm, 854 nm (the fifth light-emitting peak wavelength), 867 nm and 882 nm (the third light-emitting peak wavelength). The half-height wavelength width corresponding to the wavelength of the first light-emitting peak of the first light, the half-height wavelength width corresponding to the wavelength of the second light-emitting peak of the second light, the half-height wavelength width corresponding to the wavelength of the third light-emitting peak of the third light, the half-height wavelength width corresponding to the wavelength of the fourth light-emitting peak of the fourth light and the half-height wavelength width corresponding to the wavelength of the fifth light-emitting peak of the fifth light are greater than 0 nm and less than or equal to 60 nm, preferably between 15 nm and 50 nm, more preferably between 15 nm and 40 nm. See FIG. 4, the half-height wavelength widths corresponding to the light-emitting wavelengths of other unexplained 747 nm, 760 nm, 785 nm, 798 nm, 824 nm, 839 nm and 867 nm are also greater than 0 nm and less than or equal to 60 nm, preferably between 15 nm to 50 nm between, more preferably between 15 nm to 40 nm. In the experimental operation in the first through third embodiments of the present disclosure, the half-height wavelength width corresponding to each of the light-emitting peak wavelengths is 55 nm. When the light-emitting element is a laser diode, the half-height wavelength width corresponding to each of the light-emitting peak wavelengths is greater than 0 nm and less than or equal to 60 nm, for example, 1 nm.

The wavelength ranges of the two light-emitting diodes corresponding to the aforementioned two adjacent light-emitting peak wavelengths may not overlap. For example, in the aforementioned first embodiment, second embodiment and third embodiment, the half-height wavelength width corresponding to each light-emitting peak wavelength is 15 nm, and the width of the wavelength range corresponding to each light-emitting peak wavelength (that is, the difference between the maximum value and the minimum value of the wavelength range) is 40 nm. The wavelengths of the two adjacent light-emitting peaks differ from each other by 80 nm. For another example, if the light-emitting element is a laser diode, the half-height wavelength width corresponding to each light-emitting peak wavelength is 1 nm, the width of the wavelength range is 4 nm, and the difference between the adjacent two light-emitting peak wavelengths is 5 nm. The wavelength ranges of the two light-emitting elements (laser diodes) corresponding to the two adjacent light-emitting peak wavelengths do not overlap.

Preferably, in the first through third embodiments, an image capture device is used to detect the object (A) to be analyzed, so as to generate the spectrum of the object to be analyzed. The image capture device is a phone or pad computer. As mentioned above, the solid state light source emitter (120) can control and make a plurality of the light emitting diodes respectively present a discontinuous light emission of on-off frequencies. The on-off frequencies may be the same or different from each other, or he on-off frequencies may be partially the same or partially different. The aforementioned on-off frequencies are between 0.05 times/sec to 50,000 times/sec. Among the on-off frequencies, the time interval for turning on (lighting up) the light-emitting diode is between 0.00001 seconds and 10 seconds, and the time interval for turning off (extinguishing) the light-emitting diode in the on-off frequency is between 0.00001 seconds and 10 seconds. The period of the on-off frequency refers to the sum of the time interval for turning on (lighting up) the light-emitting diode and the time interval for turning off (extinguishing) the light-emitting diode in succession, and the period of the on-off frequency is the reciprocal of the on-off frequency. In other words, the period of the on-off frequency can be understood as the sum of the light-emitting diodes that a plurality of light-emitting diodes continuously are lighted up for a light-on time interval and immediately turned off for an extinguishing time interval without interruption, and the light-on time interval is between 0.00001 seconds and 10 seconds, the extinguishing time interval is between 0.00001 seconds and 10 seconds. Preferably, the on-off frequency is between 0.5 times/sec to 50,000 times/sec; more preferably, the on/off frequency is from 5 times/sec to 50,000 times/sec. The light-emitting diodes exhibiting discontinuous light-emitting states can greatly reduce the influence of the object (A) to be analyzed by the thermal energy of the light emitted by the light-emitting diodes, and avoid the object (A) to be analyzed containing organisms qualitatively change. Thus, it is especially suitable for the object (A) to be analyzed that is sensitive to thermal energy, and it is especially suitable for that the light of the wavelength range emitted by the light-emitting diode is the near-infrared light.

It is particularly noted that the above-mentioned light-emitting element and the image capture device and the light detector of the light receiver (121) are synchronously operated. The synchronous operation can refer to “the image capture device and the light detector operates discontinuously at an operating frequency, and the on-off frequency of the light-emitting element is the same as the operating frequency of the image capture device and the light detector of the light receiver (121)”.

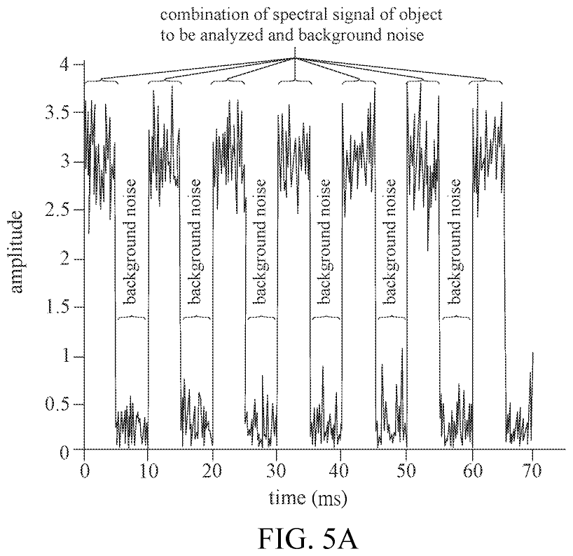

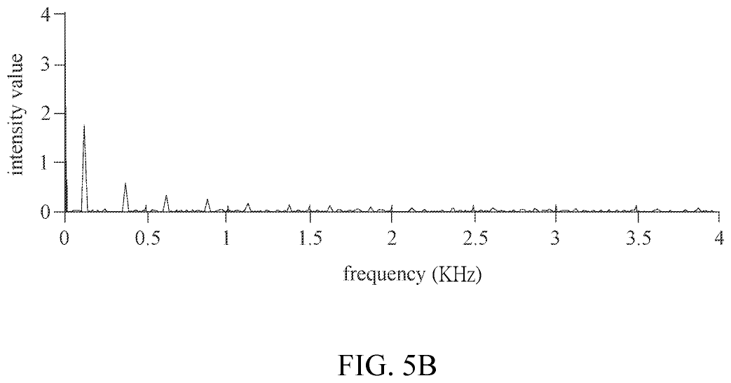

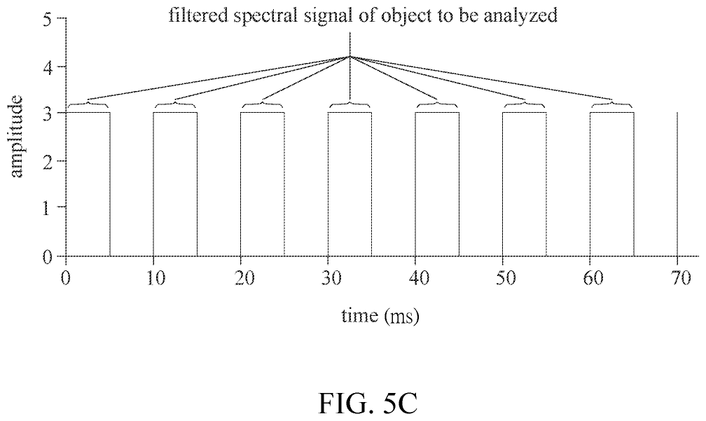

Refer to FIG. 5A, in this embodiment, the optical detection device (12) is operated in the discontinuous light emission mode of the on-off frequency to perform the detection of the object (A) to be analyzed, the time domain signal plot of FIG. 5A shows the time domain signal of the combination of a spectral signal of the object (A) and a background noise. A mathematical analysis module is disposed on the photodetector or the calculator, the mathematical analysis module is electrically or signally connected to the photodetector, or the mathematical analysis module is electrically or signally connected to the calculator. The mathematical analysis module can be in the form of software or hardware, and the signal collected by the photodetector is transmitted to the mathematical analysis module. When operating the image capture device to perform the detection of the object (A) to be analyzed to generate the spectrum of the object (A), the light-emitting diodes can be turned on or off simultaneously at the same on-off frequency, during the time interval of the on-off frequency which the light-emitting diode is turned on (lighted), the signal received by the photodetector is the combination of the spectral signal of the object (A) and a background noise, and during the time interval of the on-off frequency which the light-emitting diode is turned off (extinguished), the signal received by the photodetector is the background noise.

The spectral signal of the object (A) and the background noise collected by the light detector are sent to the mathematical analysis module, and the mathematical analysis module processes the time domain signal of the object (A) to discard the background noise. For example, the mathematical analysis module includes a time/frequency domain transforming unit, which converts the time domain signal of the object (A) into a frequency domain signal of the object (A), as shown in FIG. 5A. The time/frequency domain transforming unit may be a Fourier transforming unit for performing a Fourier transform on the time domain signal of the object (A) to generate the frequency domain signal of the object (A). FIG. 5B is a frequency domain signal plot of the transformed frequency domain signal of the object (A). The frequency domain signal of the object (A) is easily distinguished into the frequency domain signal of the spectral signal of the object (A) and the frequency domain signal of the background noise. In FIG. 5B, the frequency domain signal at the peak of 0 Hz or the frequency domain signal less than the on-off frequency is the frequency domain signal of the background noise. In FIG. 5B, except the frequency domain signal of the peak at 0 Hz (the frequency domain signal of the background noise), the remaining peak signals are the frequency domain signal of the spectral signal of the object (A). Preferably, in the frequency domain signal of the object (A), the frequency domain signal greater than or equal to the on-off frequency is the frequency domain signal of the spectral signal of the object (A). The mathematical analysis module discards the frequency domain signal of the background noise and leaves the frequency domain signal of the spectral signal of the object (A) to achieve filtering effect. Since the mathematical analysis module discards the frequency domain signal of the background noise, the frequency domain signal of the spectral signal of the object (A) that is left completely belongs to the object (A) and does not include the background signal, so compared with the conventional spectrometer, the optical detection device (12) of the present disclosure not only improves the signal-to-noise ratio of the object (A) in the spectrum, but also the optical detection device (12) of the present disclosure obtain a spectrum without background noise because the frequency domain signal of the background noise is discarded for filtering. Refer to FIG. 5A and FIG. 5B, and a microcontroller of the solid state light source emitter (120) can be electrically or signally connected to the mathematical analysis module to synchronously transmit the on-off frequency, the time interval of the on-off frequency which the light-emitting diodes are turned on (lighted up) and the time interval of the on-off frequency which the light-emitting diodes are turned off (extinguished) are sent to the mathematical analysis module, so that, when the microcontroller turns on (lights up) the light-emitting diodes connected to the microcontroller respectively according to the on-off frequency, the time interval of the on-off frequency which the light-emitting diodes are turned on (lighted up) and the time interval of the on-off frequency which the light-emitting diodes are turned off (extinguished), the mathematical analysis model corresponds the time interval of the on-off frequency which the light-emitting diodes are turned on (lighted up) to the spectral signal of the object (A), and the mathematical analysis model corresponds the time interval of the on-off frequency which the light-emitting diodes are turned off (extinguished) to the background noise.

It is particularly noted that the waveforms of the multiple light-emitting diodes exhibiting the discontinuous light emission of on-off frequencies are square waves, sine waves or negative sine waves.

In addition, the mathematical analysis module can also process the frequency domain signal of the spectral signal of the object (A) left by the filtering effect, and convert the frequency domain signal of the spectral signal of the object (A) to a filtered time domain signal of the object (A), and the time domain signal plot of the filtered time domain signal of the object (A) can be obtained, wherein the filtered time domain signal of the object (A) merely has the time domain signal of the spectral signal of the object (A) without the background noise. For example, the mathematical analysis module includes a frequency domain-time domain conversion unit (see FIG. 5B), the frequency domain time domain conversion unit may be used to perform an inverse Fourier transform on the frequency domain signal of the spectral signal of the object (A) left above to the filtered time domain signal of the object (A), wherein FIG. SC shows the time domain signal plot of the filtered time domain signal of the object (A). Comparing FIG. SA and FIG. SC, it can be clearly seen that in the filtered time domain signal diagram of the object (A) of FIG. SC, the filtered time domain signal of the object (A) only has the time domain signal of the spectral signal of the object (A) which is presented as a square wave, and there is no any background noise in the filtered time domain signal of the object (A). In other words, in FIG. SC, the background signal is zero, so if the value of the spectral signal of the object (A) is divided by the value of the background signal, the resulting signal-to-noise ratio will be infinite. Therefore, the present disclosure enhances the signal-to-noise ratio in the spectrogram of the test result of the object (A) to be analyzed, which can achieve the effect of accurate test. It is particularly noted that the mathematical analysis module, the time-domain frequency-domain conversion unit, and the frequency-domain time-domain conversion unit may be software or hardware types, or a combination of the above software or hardware types, wherein the mathematical analysis module, the time-domain frequency-domain conversion unit, and the frequency-domain time-domain conversion unit are electrically or signally connected to each other.

In the embodiment of the present disclosure, the wavelength range of the light source of the solid state light source emitter (120) is between 400 nm and 1700 nm, because the different groups contained in the object (A) and the same group have different wavelengths for the absorption of the light source in different physical and chemical environments. The user can adjust the wavelength range of the light source in a specific range according to the groups contained in different objects (A), so as to facilitate the analysis of the object (A).

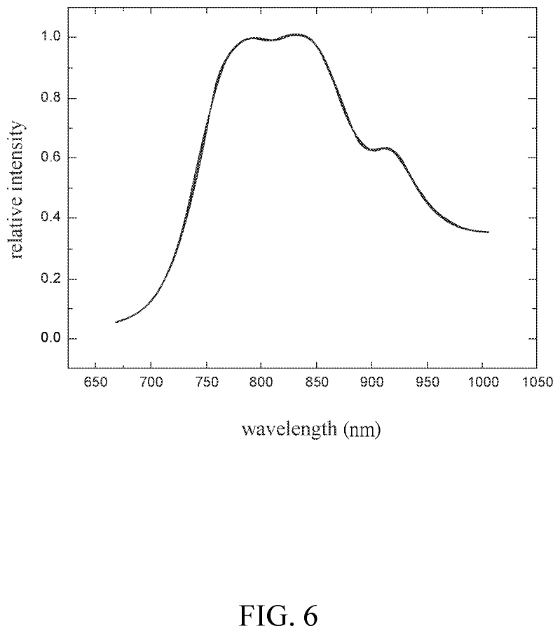

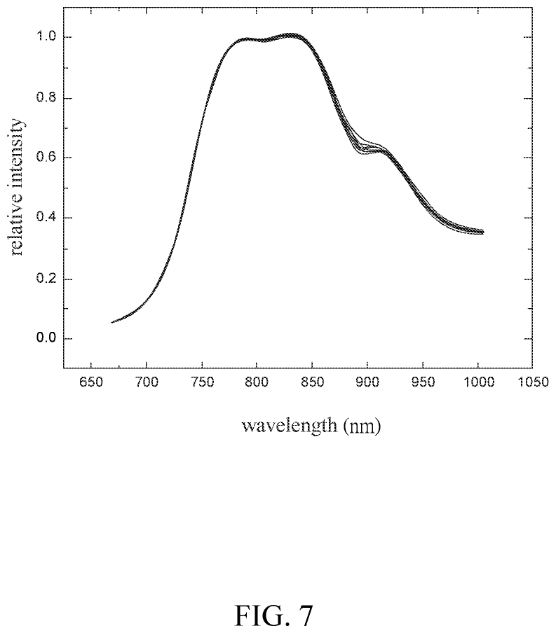

Refer to FIG. 6, and FIG. 6 is a spectrum of wheat detected by a composition analyzer of the present disclosure. Through the rotation of the accommodating device (10) of the present disclosure in the YZ plane, the grains to be analyzed can be mixed uniformly for multiple detections, and the detected data of the grains has high measurement accuracy. Further, refer to FIG. 7, and FIG. 7 is a spectrum of wheat detected by a composition analyzer of the prior art. The conventional composition analyzer mixes the detected grains by horizontal rotation of the accommodating device (10), and then performs the detection, wherein the horizontal refers to horizontal to an XY plane defined by the X direction and the Y direction. As shown in FIG. 6 and FIG. 7, the abscissa axis is wavelength, the unit is nm, and the ordinate axis is relative intensity. In this test, there are many composition analyzers in the continuous wavelength range between about 650 nm and 1000 nm, and spectral measurements are performed on grains several times. As shown in FIG. 6, the results of the relative intensity distribution of light in each spectral measurement tended to be consistent. In contrast, as shown in FIG. 7, the results of the relative intensity distribution of light in each spectral measurement are not nearly the same, especially in the continuous wavelength range between 800 nm and 1000 nm.

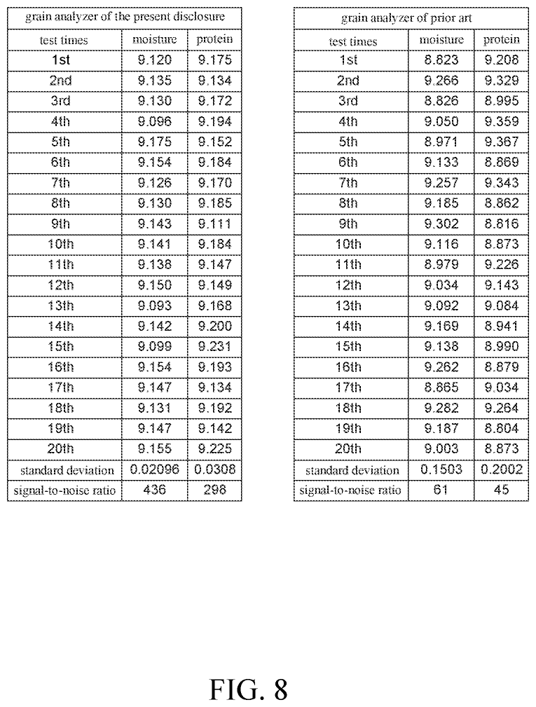

Refer to FIG. 8, and FIG. 8 is a composition comparison analysis table chart of the spectrums of wheat detected by the composition analyzers of the present disclosure and the prior art.

The table records the test number of each of multiple detections and the moisture and protein data of the grains detected by the composition analyzer, as well as the standard deviation and signal-to-noise ratio of the data. The table shows that the standard deviation value of protein measured by the present disclosure is 0.0308 and the standard deviation value of moisture measured by the present disclosure is 0.02096, while the standard deviation value of protein measured by the conventional method is 0.2002 and the standard deviation value of moisture measured by the conventional method is 0.1503 Therefore, compared with the conventional composition analyzer, the standard deviation value of the present disclosure is significantly smaller, which means that the value of each detection is not much different from the average value. Furthermore, the table shows that the signal-to-noise ratio of the protein measured by the present disclosure is 298 and the signal-to-noise ratio of water measured by the present disclosure is 436, while the signal-to-noise ratio of the protein measured by the conventional method is 45 and the signal-to-noise ratio of water measured by the conventional method is 61. Therefore, compared to the conventional composition analyzer, the detected signal-to-noise ratio of the present disclosure is also significantly higher, which means that the present disclosure can indeed improve the test accuracy.

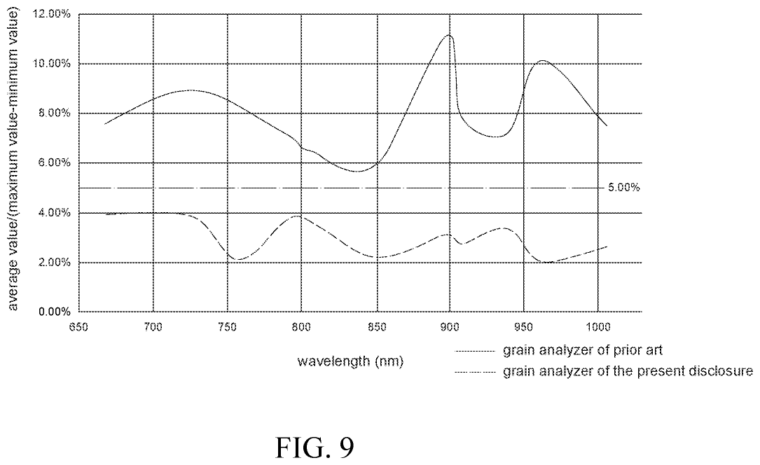

Refer to FIG. 9, and FIG. 9 is a comparison analysis table chart of the spectrums of the object to be analyzed which is detected by the composition analyzers of the present disclosure and the prior art. The abscissa axis is wavelength, the unit is nm. The ordinate axis is the specific value obtained after multiple measurements, wherein the specific value is the maximum value minus the minimum value and then divided by the average value obtained after multiple measurements, and the unit is percentage. In this experiment, the spectrum of grains is measured multiple times with the composition analyzer in the continuous wavelength range between about 650 nm and 1000 nm, and the percentage of the abscissa axis is 5.00% as the standard. If the specific value is higher than 5.00%, the difference between the maximum value and the minimum value will be greater, and it means that the measurement results of the composition analyzer (1000) have lower accuracy, and vice versa, if the difference between the maximum value and the minimum value is smaller, the accuracy between each measurement result of the composition analyzer (1000) will be higher. As shown in FIG. 9, it can be clearly observed that the specific values of the results measured by the composition analyzer of the present disclosure are all below 5.00%, while the specific values of the results measured by the conventional composition analyzer are all higher than 5.00%.

To sum up, as shown in FIG. 6 to FIG. 9, through the rotation of the accommodating device (10) of the present disclosure in the YZ plane, the grains to be analyzed can be mixed uniformly for multiple detections, and the detected data of the grains has high measurement accuracy.

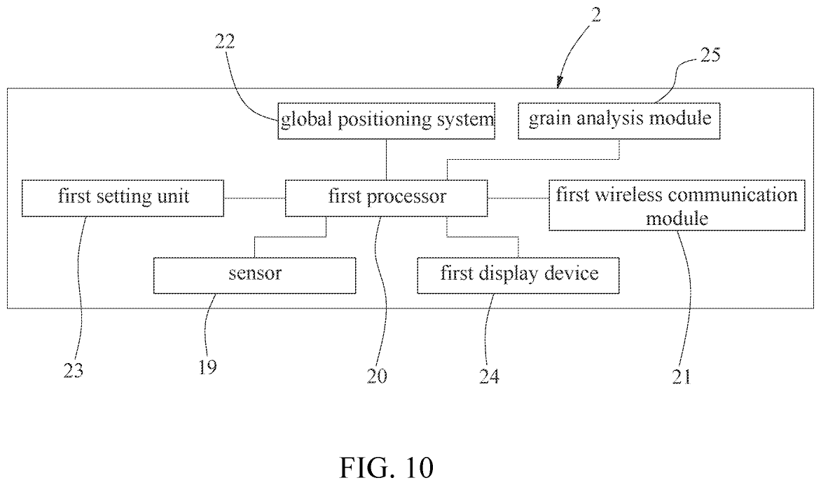

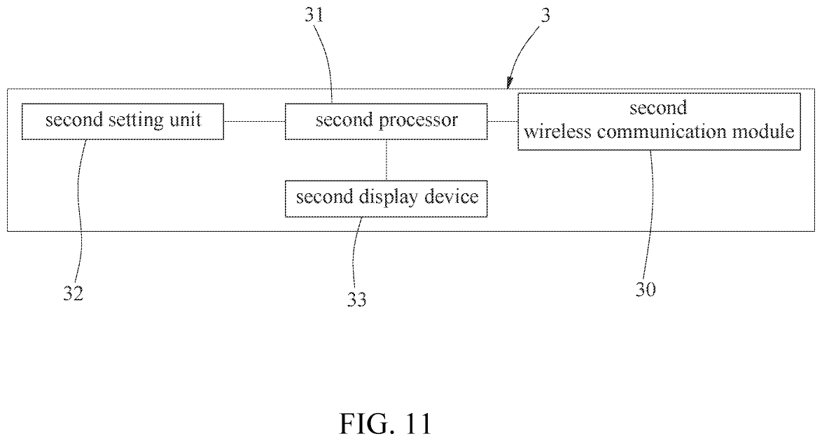

Refer to FIG. 10 and FIG. 11, FIG. 10 is a block diagram of a composition analysis system according to an embodiment of the present disclosure, and FIG. 11 is a block diagram of an electronic device according to an embodiment of the present disclosure. Another objective of the present disclosure is to provide a composition analysis system which adopts a composition analyzer (1000). The composition analysis system (2) comprises an accommodating device (10), an optical detection device (12), a driving device (13), at least a support part (14) and a first processor (20). The accommodating device (10) comprises an accommodating space (101), two transparent plates (102) and a rotating part (11). The transparent plates (102) are disposed on one of two opposite sides of the accommodating space (101). The rotating part (11) is arranged throughout the accommodating device (10) or is arranged on both sides of the accommodating device (10). An extending direction of the rotating part (11) is defined as an X direction, the X direction is different from a Y direction and a Z direction, the Y direction and the Z direction form a YZ plane, the accommodating device (10) is configured to rotate in the YZ plane, a normal line of the YZ plane and the X direction have an angle therebetween, and the angle is larger than or equal to 0 degrees and less than 90 degrees. The optical detection device (12) has a solid state light source emitter (120) and a light receiver (121), wherein the solid state light source emitter (120) has a light source, the light receiver (121) receives a light beam emitted from the light source, and the solid state light source emitter (120) and the light receiver (121) are respectively disposed on positions adjacent to the two opposite sides of the accommodating device (10). The driving device (13) is connected to the rotating part (11). The support part (14) is pivotally connected to the rotating part (11). The first processor (20) is electrically connected to the optical detection device (12), the driving device (13), a grain analysis module (25), a first wireless communication module (21) and a global positioning system (22).

The grain analysis module (25) can analyze the spectrogram after the optical detection device (12) detects the grain, so as to analyze the values of water, protein and gray matter of the grain, which can be used to further identify the grade and quality of the grain. Take wheat as an example, when wheat is processed into flour, its protein content will affect water absorption or gluten strength. The gray matter content can be used to evaluate the expected output value of wheat after milling, and the moisture content of wheat itself is also important, which affects the amount of water added when processing the wheat into flour. It is particularly noted that the grain analysis module (25) of the present disclsoure is not limited to only analyze parameters such as water, protein, and gray matter of the above-mentioned grains, but can also analyze the proportions or contents of other components of the grains as required.

In one embodiment of the present disclosure, the first wireless communication module (21) is communicatively connected to a second wireless communication module (30) of an electronic device (3), and the second wireless communication module (30) is electrically connected to the first processor (31). The electronic device (3) can be a personal computer, a personal mobile communication device, a notebook computer or a tablet computer, and the like.

In practice, the composition analyzer (1000) can transmit the values such as moisture, protein and gray matter of the grain analyzed by the grain analysis module (25) to an electronic device (3) through the first wireless communication module (21). To allow the user to access the values of water, protein and gray matter of the grain at any time through the electronic device (3), the first wireless communication module (21) and the second wireless communication module (30) can be selected from Wi-Fi, WiMAX, IEEE 802.11 series, 4G network, 5G network, HSPA network, LTE network or Bluetooth.

In one embodiment of the present disclosure, this composition analysis system (2) further comprises a sensor (19), and this sensor (19) is electrically connected to this first processor (20). The sensor (19) may comprise a relative humidity sensor or a temperature sensor or both, the relative humidity sensor is used to sense a relative humidity in the air and generate a relative humidity data, and the temperature sensor is used to sense an ambient temperature during plant growth, and generate an ambient temperature data. According to the relative humidity or the ambient temperature, the growth of the grain can be predicted, and the composition analyzer (1000) can transmit the information sensed by the sensor (19) to an electronic device (1000) through the first wireless communication module (21). The information can be the ambient temperature data sensed by the temperature sensor and the relative humidity data sensed by the relative humidity sensor, so that the user can access the ambient temperature data and the relative humidity data when the grain grows through the electronic device (3) at any time.

In one embodiment of the present disclosure, the composition analysis system (2) further includes a first setting unit (23), the first setting unit (23) can be exemplified but not limited to a touch screen or a button, and the first setting unit (23) is electrically connected to the first processor (20). The first setting unit (23) can input any parameters of the planted crop, such as crop information, a crop type, a record date, an analysis area (R) or a crop harvesting plan, so that the user can directly operate to adjust parameters of the planted crop via the composition analyzer (1000).

In one embodiment of the present disclosure, the composition analysis system (2) further comprises a first display device (24), and the first display device (24) is electrically connected to the first processor (20). The first display device (24) can display the spectrogram generated by the optical detection device (12), the operating speed or frequency of the driving device (13), values which the grain analysis module (25) analyzes the spectrogram, the information generated by the positioning information (P) of the global positioning system (22), the crop information, the crop type, the record date, the analysis area (R) or the crop harvesting plan or the information benefit to the user for judging and analyzing. The first display device (24) can be a liquid crystal screen.

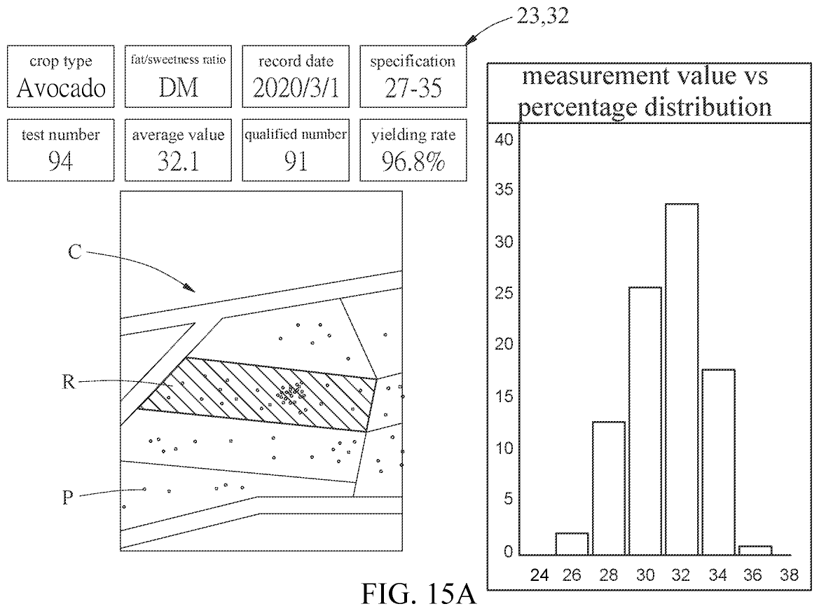

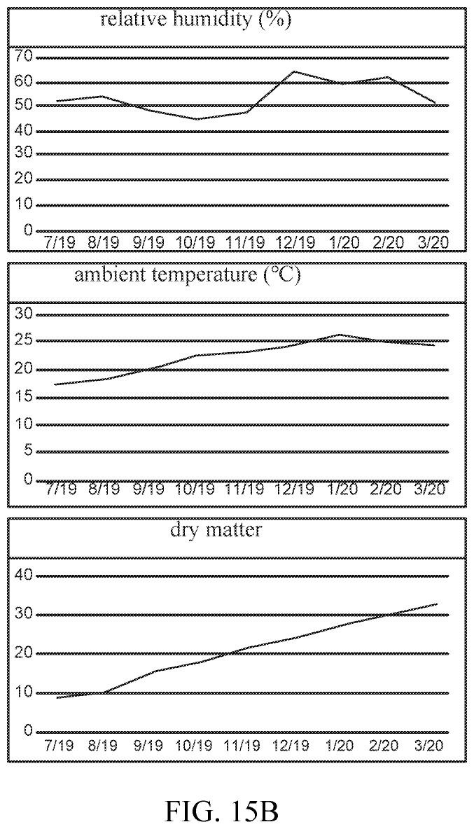

In one embodiment of the present disclosure, the electronic device (3) further comprises a second setting unit (32), the second setting unit (32) is electrically connected to the second processor (31), and the second setting unit (32)) can be exemplified but not limited to touch screens or buttons. The second setting unit (32) can input crop information, a crop type, a record date, an analysis area (R) or a crop harvesting plan and any crop-related parameters. The second setting unit (32) can input any parameters of the planted crop, such as crop information, a crop type, a record date, an analysis area (R) or a crop harvesting plan. In actual implementation, as shown in FIG. 15A and FIG.15B, the crop type is the planted crop species, and the record date is the date when the crop is detected. The crop information can be the relative humidity (RH %) or temperature of the environment (degrees in Celsius); or the crop information can be the specification of dry matter content, moisture content, protein content and fat/sweetness ratio, etc. in the crop. The specification is a preset crop quality standard value, which can be based on the above-mentioned dry matter (dry matter) content, water content, protein content and fat/sweetness ratio, etc. or other parameters that are conducive to evaluating crop quality standards which can be the basis for a single-parameter judgment or comprehensive judgment. The number of tests is the number of times the crop is detected by the composition analyzer. The composition analyzer produces a crop quality value after each test of the crop, and the average value is the sum of all tested crop quality values divided by the total number of tests. The qualified number is counted once every time the crop quality value meets the specification. The yielding rate is the qualified number divided by the number of tests. The crop harvest plan can predict the harvest date of the crop based on the crop information and crop type.

In one embodiment of the present disclosure, the electronic device (3) further comprises a second display device (33), the second display device (33) is electrically connected to the second processor (31), the second display device (33)) can display the spectrogram generated by the optical detection device (12), the operating speed or frequency of the driving device (13), the value analyzed by the grain analysis module (25) on the spectrogram and the positioning information (P) generated by the global positioning system (22), etc., or crop information, a crop type, a record date, an analysis area (R) or a crop harvesting plan (i.e. parameter related to the planted crop), or any information that is beneficial to the user's judgment and analysis. The second display device (33) can be a liquid crystal screen.

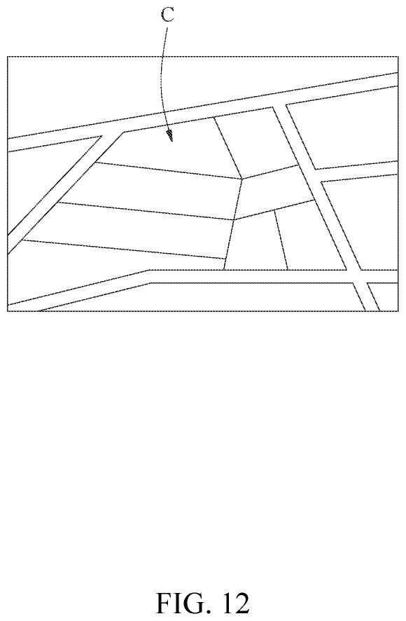

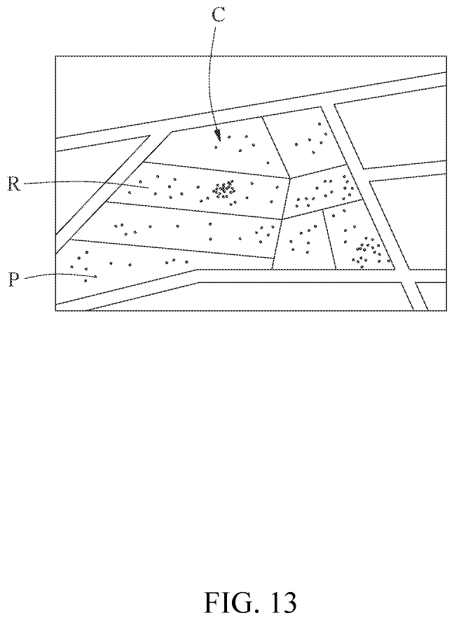

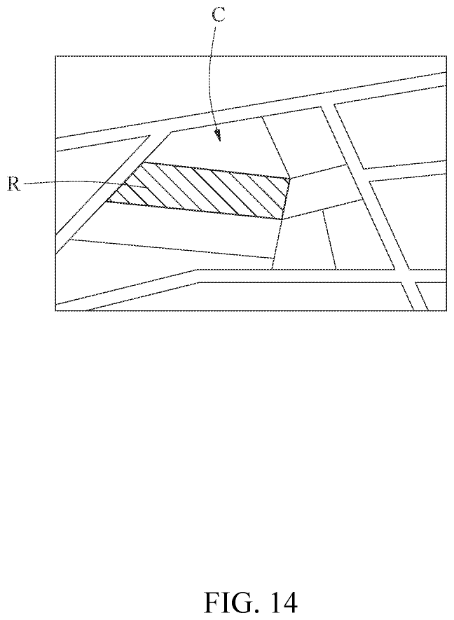

Refer to FIG. 12 to FIG. 14, FIG. 12 a schematic diagram of a crop origin according to an embodiment of the present disclosure, FIG. 13 is a first schematic diagram of an analysis area according to an embodiment of the present disclosure, and FIG. 14 is a second schematic diagram of an analysis area according to an embodiment of the present disclosure. The global positioning system (22) can provide an accurate three-dimensional positioning function, and the user can locate positioning information (P) for a crop origin (C) through the global positioning system (22). The positioning information (P) can be the coordinates of longitude, latitude and altitude of the location. In actual implementation, when the user uses the composition analyzer to detect grains, in addition to generating the values of water, protein and gray matter of the grains, the global positioning system (22) also locates the corresponding position of the detected position. Then, the first processor (20) transmits the moisture, protein and gray matter values of the grain detected by the composition analyzer (1000) and the positioning information (P) to the second wireless communication module (30) via the first wireless communication module (21). The user can refer to the positioning information (P) and the corresponding values, crop information or crop types as a basis for formulating crop harvesting plans in the future. The user can also use the first setting unit (23) or the second setting unit (32) to set analysis area to be analyzed, and the analysis area (R) may contain multiple or single pieces of the positioning information (P), as shown in FIG. 15A and FIG. 15B, so that any crop-related parameters such as crop information, crop type, record date or crop harvesting plan of the analysis area (R) can be obtained. The crop type is the planted crop species, and the record date is the date when the crop is detected. The crop information can be the relative humidity (RH %) or temperature of the environment (degrees in Celsius); or the crop information can be the specification of dry matter content, moisture content, protein content and ratio of oil/sweetness, etc. in the crop. The specification is a preset crop quality standard value, which can be based on the above-mentioned dry matter (dry matter) content, water content, protein content and fat/sweetness ratio, etc. or other parameters that are conducive to evaluating crop quality standards which can be the basis for a single-parameter judgment or comprehensive judgment. The number of tests is the number of times the crop is detected by the composition analyzer. The composition analyzer produces a crop quality value after each test of the crop, and the average value is the sum of all tested crop quality values divided by the total number of tests. The qualified number is counted once every time the crop quality value meets the specification. The yielding rate is the qualified number divided by the number of tests. The crop harvest plan can predict the harvest date of the crop based on the crop information and crop type.

To sum up, compared with prior art and product, the present disclosure has one of the advantages as follows.

One objective of the present disclosure is to utilize the rotation of the accommodating device in the YZ plane via the composition analyzer of the present disclosure, so that the grains to be analyzed can be detected under the condition of uniform mixing, and the amount of multiple repeated measurements is achieved to obtain data of the composition of the grains.

One of the objectives of the present disclosure is to measure not only grains via the structure of the accommodating device, but also to measure the object to be analyzed, such as, granules, powders, Chinese herbal medicines, liquids or fluids, and measured values of various types are used to classify and filter out the object to be analyzed.

One of the objectives of the present disclosure is to reduce the possibility of contamination of grain to be analyzed, which is caused by that samples need to be taken out and mixed again when repetitive measurement operations are performed manually in the past through the configuration relationship and rotation manner between the composition analyzer structures of the present disclosure, so that the conditional factors of the previous and subsequent measurements are similar to each other.

Any embodiment or claim scope of the present disclosure need not achieve all the purposes or advantages or features disclosed in the present disclosure. In addition, the terms such as “first” and “second” mentioned in this specification or the claim scope of the present disclosure are only used to name the elements or to distinguish different embodiments or ranges, and are not used to limit the number of elements.

What is claimed is:

1. A composition analyzer, comprising:
an accommodating device, configured to accommodate an object to be analyzed, and having an accommodating space, a transparent plate and a rotating part, wherein the transparent plate is disposed on one of two opposite sides of the accommodating space, the rotating part is disposed on the accommodating device, an extending direction of the rotating part is defined as an X direction, the X direction is different from a Y direction and a Z direction, the Y direction and the Z direction form a YZ plane, the accommodating device is configured to rotate in the YZ plane, a normal line of the YZ plane and the X direction have an angle therebetween, and the angle is larger than or equal to 0 degrees and less than 90 degrees;
an optical detection device, having a solid state light source emitter and a light receiver, wherein the solid state light source emitter has a light source, the light receiver receives a light beam emitted from the light source, and the transparent plate is provided to the light beam for passing through, wherein another one of the two opposite side of the accommodating space is disposed with one of another one transparent plate, a light reflection plate and a non-transparent plate;
a driving device, connected to the rotating part; and
at least a support part, pivotally connected to the rotating part.

2. The composition analyzer of claim 1, wherein when the other one of the two opposite side of the accommodating space is disposed with other one transparent plate, the solid state light source emitter and the light receiver are respectively disposed on positions adjacent to the two opposite sides of the accommodating device.

3. The composition analyzer of claim 1, wherein when the other one of the two opposite side of the accommodating space is disposed with the light reflection plate, the solid state light source emitter and the light receiver are disposed positions adjacent to the one of the two opposite sides of the accommodating device.

4. The composition analyzer of claim 1, wherein when the other one of the two opposite side of the accommodating space is disposed with the a non-transparent plate, the solid state light source emitter is disposed on a position adjacent to the one of the two opposite sides of the accommodating device, and the light receiver is disposed on a position adjacent to the other one of the two opposite sides of the accommodating device and facing to the one of the two opposite sides of the accommodating device.

5. The composition analyzer of claim 1, wherein the accommodating device further comprises a cover and an opening, the opening is communicated to the accommodating space, and the cover is configured to movably close the opening.

6. The composition analyzer of claim 5, wherein the cover is sieved to the opening.

7. The composition analyzer of claim 5, wherein the cover is pivotally connected to the accommodating device via a first pivot.

8. The composition analyzer of claim 1, wherein material of the transparent plate comprises glass, sapphire, quartz or acrylic.

9. The composition analyzer of claim 1, wherein a wavelength the light source of the solid state light source emitter is ranged from 180 nm to 2500 nm.

10. The composition analyzer of claim 1, a wavelength the light source of the solid state light source emitter is ranged from 400 nm to 1700 nm.

11. The composition analyzer of claim 1, wherein a cross-sectional shape of the accommodating device is a circle, an ellipse, a polygon or an irregular shape.

12. The composition analyzer of claim 1, wherein the composition analyzer is disposed in an interior of a case.

13. The composition analyzer of claim 12, wherein the case further comprises a cover, and the cover is pivotally connected to the case via a second pivot.

14. The composition analyzer of claim 1, wherein the case further comprises at least a heat dissipation hole disposed thereon.

15. The composition analyzer of claim 13, wherein the case further comprises a heat dissipation unit disposed in the interior of the case.

16. The composition analyzer of claim 1, further comprising a sensor.

17. The composition analyzer of claim 16, wherein the sensor comprises at least one of a relative humidity sensor and a temperature sensor.

18. A composition analysis system, adapted to a composition analyzer, comprising:
an accommodating device, configured to accommodate an object to be analyzed, and having an accommodating space, a transparent plate and a rotating part, wherein the transparent plate is disposed on one of two opposite sides of the accommodating space, the rotating part is disposed on the accommodating device, an extending direction of the rotating part is defined as an X direction, the X direction is different from a Y direction and a Z direction, the Y direction and the Z direction form a YZ plane, the accommodating device is configured to rotate in the YZ plane, a normal line of the YZ plane and the X direction have an angle therebetween, and the angle is larger than or equal to 0 degrees and less than 90 degrees;
an optical detection device, having a solid state light source emitter and a light receiver, wherein the solid state light source emitter has a light source, the light receiver receives a light beam emitted from the light source, and the transparent plate is provided to the light beam for passing through, wherein another one of the two opposite side of the accommodating space is disposed with one of another one transparent plate, a light reflection plate and a non-transparent plate;
a driving device, connected to the rotating part;
at least a support part, pivotally connected to the rotating part; and a a first processor, electrically connected to the optical detection device, the driving device, a grain analysis module, a first wireless communication module and a global positioning system.

19. The composition analysis system of claim 18, further comprising a sensor electrically connected to the first processor.

20. The composition analysis system of claim 19, wherein the sensor comprises at least one of a relative humidity sensor and a temperature sensor.

21. The composition analysis system of claim 18, further comprising a first setting unit electrically connected to the first processor, wherein the first setting unit is used to input at least one parameter related to a planted crop.

22. The composition analysis system of claim 21, wherein the at least one parameter related to the planted crop comprises at least one of crop information, a crop kind, a record date, an analysis area and a crop harvesting plan.

23. The composition analysis system of claim 18, further comprising a first display device electrically connected to the first processor.

24. The composition analysis system of claim 18, wherein the first wireless communication module is communicatively connected to a second wireless communication module of an electronic device, and the second wireless communication module is electrically connected to a second processor.

25. The composition analysis system of claim 24, wherein the electronic device further comprises a second setting unit electrically connected to the second processor, wherein the second setting unit is used to input at least one parameter related to a planted crop.

26. The composition analysis system of claim 25, wherein the at least one parameter related to the planted crop comprises at least one of crop information, a crop kind, a record date, an analysis area and a crop harvesting plan.

27. The composition analysis system of claim 24, wherein the electronic device further comprises a second display device electrically connected to the second processor.
